(19) United States
(12) Patent Application Publication
(10) Pub. No.: US 2023/0012706 A1
(43) Pub. Date: Jan. 19, 2023

(54) BUOYANT HOLLOW PARTICLES COMPOSTION AND METHOD

(71) Applicant: 3M INNOVATIVE PROPERTIES COMPANY, St. Paul, MN (US)

(72) Inventors: Katie F. Wlaschin, St. Paul, MN (US); Federica Sgolastra, St. Paul, MN (US); Masayuki Nakamura, Woodbury, MN (US)

(21) Appl. No.: 17/785,332

(22) PCT Filed: Dec. 17, 2019
(86) PCT No.: PCT/IB2019/060937

§ 371 Date: Jun. 14, 2022

(51) Int. Cl. C12N 11/14; C12N 5/00

(52) U.S. Cl. CPC C12N 11/14; C12N 5/0075; C12N 2533/12; C12N 2531/00

(57) ABSTRACT

A composition and a method for culturing cells. The composition includes a plurality of buoyant hollow particles, the buoyant hollow particles comprising a siliceous surface; and a plurality of mammalian cells attached to the siliceous surface of the buoyant hollow particles; wherein the buoyant hollow particles are less dense than a media; and wherein the average seeding density is 3-50 adherent cells/buoyant hollow particle.

### FIELD

The present disclosure relates to compositions and methods useful for cell culture.

### BACKGROUND

Advances in personalized medicine and gene editing in mammalian cells (specifically CRISPR technology) has opened the opportunity for use of cultured cells as a treatment for disease. In these applications, the cells themselves are the therapeutic agent. Most cells that are useful for these applications are anchorage dependent. Anchorage dependent cells require attachment to a surface for survival, proliferation, and maintenance of the desired phenotype. There is a need for better cell culture composition.

### SUMMARY

Thus, in one aspect, the present disclosure provides a composition. The composition includes a plurality of buoyant hollow particles, the buoyant hollow particles comprising a siliceous surface; and a plurality of mammalian cells attached to the siliceous surface of the buoyant hollow particles; wherein the buoyant hollow particles are less dense than a media; and wherein the average seeding density is 3-50 adherent cells/buoyant hollow particle.

In another aspect, the present disclosure provides a method for culturing cells. The method includes providing a plurality of buoyant hollow particles, the buoyant hollow particles comprising a siliceous surface, wherein the buoyant hollow particles are less dense than a media; contacting the buoyant hollow particles with a media comprising a plurality of mammalian cells; allowing the mammalian cells to attach to the siliceous surface of the buoyant hollow particles; and culturing the mammalian cells on the siliceous surface of the buoyant hollow particles by agitation, to yield the cell culture.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### DEFINITIONS

For the following defined terms, these definitions shall be applied for the entire Specification, including the claims, unless a different definition is provided in the claims or elsewhere in the Specification based upon a specific reference to a modification of a term used in the following definitions:

As used herein, the term “bubble,” refers to a small, hollow globule, for example, a small spherical volume of gas encased within a thin film.

As used herein, the term “analyte,” refers to any substance which may be present in a sample, and that it is desirable to separate from the sample or to detect in an assay. The analyte can be, without limitation, any substance. For example, an analyte may comprise a substance for which there exists a naturally occurring antibody or for which an antibody can be prepared. The analyte may, for example, be a protein, a polypeptide, a hapten, a carbohydrate, a lipid, a drug, a bacterium, a virus, an enzyme, a cell, a cellular subcomponent or organelle (e.g., lysozomes, mitochondria) or any other of a wide variety of biological or non-biological molecules, complexes or combinations thereof. In still another example, the analyte is a nucleic acid (DNA, RNA, PNA and nucleic acids that are mixtures thereof or that include nucleotide derivatives or analogs).

As used herein, the term “alkyl” refers to a monovalent group that is a radical of an alkane and includes groups that are linear, branched, cyclic, bicyclic, or a combination thereof. The alkyl group typically has 1 to 30 carbon atoms. In some embodiments, the alkyl group contains 1 to 20 carbon atoms, 1 to 10 carbon atoms, 1 to 6 carbon atoms, 1 to 4 carbon atoms, or 1 to 3 carbon atoms. In some embodiments, the alkyl group contains one or more heteroatoms, such as oxygen, nitrogen, or sulfur atoms.

As used herein, the term “alkylene” refers to a divalent group that is a radical of an alkane and includes groups that are linear, branched, cyclic, bicyclic, or a combination thereof. The alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. In some embodiments, the alkylene group contains one or more heteroatoms, such as oxygen, nitrogen, or sulfur atoms.

As used herein, the term “alkyleneoxy” refers to a divalent group that is an oxy group bonded directly to an alkylene group.

As used herein, the term “alkoxy” refers to a monovalent group having an oxy group bonded directly to an alkyl group.

As used herein, the term “aryl” refers to a monovalent group that is aromatic or heteroaromatic. The aryl has at least one unsaturated carbocylic or heterocyclic ring and can have one or more additional fused rings that can be unsaturated, partially saturated, or saturated. Aryl groups often have 6 to 20 carbon atoms, 6 to 18 carbon atoms, 6 to 16 carbon atoms, 6 to 12 carbon atoms, or 6 to 10 carbon atoms, and 0 to 5 heteroatoms selected from oxygen, sulfur, or nitrogen.

As used herein, the term “arylene” refers to a divalent group that is aromatic or heteroaromatic. The arylene has at least one unsaturated carbocylic or heterocyclic ring and can have one or more additional fused rings that can be unsaturated, partially saturated, or saturated. Arylene groups often have 6 to 20 carbon atoms, 6 to 18 carbon atoms, 6 to 16 carbon atoms, 6 to 12 carbon atoms, or 6 to 10 carbon atoms, and 0 to 5 heteroatoms selected from oxygen, sulfur, or nitrogen.

As used herein, the term “aryloxy” refers to a monovalent group having an oxy group bonded directly to an aryl group.

As used herein, the term “aralkyl” refers to a monovalent group that is an alkyl group substituted with an aryl group. Aralkyl groups often have an alkyl portion with 1 to 10 carbon atoms, 1 to 6 carbon atoms, or 1 to 4 carbon atoms and an aryl portion with 6 to 20 carbon atoms, 6 to 18 carbon atoms, 6 to 16 carbon atoms, 6 to 12 carbon atoms, or 6 to 10 carbon atoms.

As used herein, the term “aralkyloxy” refers to a monovalent group having an oxy group bonded directly to an aralkyl group. Equivalently, it can be considered to be an alkoxy group substituted with an aryl group.

As used herein, the term “aralkylene” refers to a divalent group that is an alkylene group substituted with an aryl group or an alkylene group attached to an arylene group. Aralkylene groups often have an alkylene portion with 1 to 10 carbon atoms, 1 to 6 carbon atoms, or 1 to 4 carbon atoms and an aryl or arylene portion with 6 to 20 carbon atoms, 6 to 18 carbon atoms, 6 to 16 carbon atoms, 6 to 12 carbon atoms, or 6 to 10 carbon atoms.

As used herein, the term “acyloxy” refers to a monovalent group of formula —O(CO)R^{b} where R^{b} is alkyl, aryl, or aralkyl. Suitable alkyl R^{b} groups often have 1 to 10 carbon atoms, 1 to 6 carbon atoms, or 1 to 4 carbon atoms. Suitable aryl R^{b} groups often have 2 to 12 carbon atoms and 0 to 3 heteroatoms, such as, for example, phenyl, furyl, or imidazolyl. Suitable aralkyl R^{b} groups often have an alkyl group with 1 to 10 carbon atoms, 1 to 6 carbon atoms, or 1 to 4 carbon atoms that is substituted with an aryl having 6 to 12 carbon atoms such as, for example, phenyl.

As used herein, the term “hydrolyzable group” refers to a group that can react with water having a pH of 1 to 10 under conditions of atmospheric pressure. The hydrolyzable group is often converted to a hydroxyl group when it reacts. The hydroxyl group often undergoes further reactions. Typical hydrolyzable groups include, but are not limited to, alkoxy, aryloxy, aralkyloxy, acyloxy, or halo. As used herein, the term is often used in reference to one of more groups bonded to a silicon atom in a silyl group.

As used herein, the term “non-hydrolyzable group” refers to a group that cannot react with water having a pH of 1 to 10 under conditions of atmospheric pressure. Typical non-hydrolyzable groups include, but are not limited to alkyl, aryl, and aralkyl.

As used herein, the term “seeding density” refers to the number of cells per unit (for example, 1 buoyant hollow particle).

As used herein, the span value was calculated from the 10P, 50P and 90P values using the formula, span =(90P-10P)/50P. The size for which 10 percent of the particles in the distribution were smaller (10P), the size for which 50 percent of the particles in the distribution were smaller (50P), and the 3 5 size for which 90 percent of the particles in the distribution were smaller (90P) were determined from the particle size distribution.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure relates to compositions for use in culture of adherent cells. The compositions may include a plurality of buoyant hollow particles. The buoyant hollow particles may have a siliceous surface. The compositions may include a plurality of cells attached to the siliceous surface of the buoyant hollow particles.

Use of floating buoyant hollow particles enables easy separation of cells from the carriers. It was strongly held that microcarriers must be denser than water to maintain the cells in contact with the media and achieve the use of the reactor volume (Merlin O-W. 2015. Phil. Trans. R. Soc. B370). However, the current application shows that agitation can also be used to “sink” buoyant microcarriers sufficiently to allow for use of the cell culture volume. Use of any microcarrier can address the area limitation for expansion of attachment cells, but the added feature of buoyancy can potentially reduce the labor and need for specialized equipment, which may be desirable for scaling up multiple parallel processes. An advantage of using buoyant hollow particles over sinking microcarriers is that after the cells are detached from the carrier (e.g. by trypsinization or other methods) it allows for easy separation of the cells from the particles without filtration or gradient centrifugation.

In various embodiments, buoyant hollow particles useful in the compositions of the present disclosure may include hollow particles having an at least partially solid outer region (e.g., shell) and a hollow inner region (e.g., core). For example, in some embodiments, useful particles may include bubbles having a substantially spherical hollow inner region encased by an outer region. The hollow inner region of the bubbles may be void of fluid, or be filled with a gas, including, but not limited to oxygen, nitrogen, carbon dioxide, helium, fluorocarbon gases and various combinations thereof, such as air. The outer region may be any material that can encase a volume of fluid, for example, a solid such as a metal, glass, ceramic, or similar material. In some embodiments, the outer region may include a siliceous material having a siliceous surface (e.g., for bonding to a surface-modifying agent). In one embodiment, the hollow particles may include glass bubbles, such as those sold by 3M under the trade designation SCOTCHLITE Glass Bubbles.

Generally, the buoyant hollow particles of the present disclosure may be configured and/or sized to be less dense than a media, for example, a cell culture media comprising water (e.g., based on buoyancy forces). The particles may have an average density of less than about 1 g/ml, less than about 0.8 g/ml, less than about 0.6 g/ml, or even less than about 0.4 g/ml. In some embodiments, the particles may have an average density in a range of from about 0.05 g/ml to about 0.8 g/ml, or from about 0.08 g/ml to about 0.4 g/ml. The particles may have a mean particle size of less than about 200 micrometers, less than about 120 micrometers less than about 100 micrometers, or even less than about 80 micrometers.

In illustrative embodiments, the buoyant hollow particles may have a mean particle size in a range of from about 5 to 250 micrometers, from about 10 to 120 micrometers from about 10 to 100 micrometers, or from about 20 to 80 micrometers.

In some embodiments, the siliceous surface of the buoyant hollow particles can be functionalized, coated or treated to provide a surface for growth of mammalian cells. The siliceous surface of the buoyant hollow particles can be coated with a polymer, a signaling peptide or an antibody or receptor, a surface-modifying agent or a substrate. Suitable substrate can include collagen, fibrin or other proteins. In some embodiments, the siliceous surface of the buoyant hollow particles can be charge modified, for example, plasma treated.

Generally, the surface-modifying agents of the present disclosure may include any molecules capable of coupling to particles useful for bioseparation (e.g., via covalent interactions, ionic interactions, hydrophobic interactions, or combinations thereof), and following such particle coupling, coupling to one or more analytes (e.g., via covalent interactions, ionic interactions, hydrophobic interactions, or combinations thereof) such that the analytes may be separated from a sample. In various embodiments, the surface-modifying agents of the present disclosure may include at least a binding segment, a linking segment, and a reactive segment:

Binding Segment—Linking Segment—Reactive Segment

Generally, the binding segment may include any segment capable of bonding the surface-modifying agent to the particles. The bond may be achieved, for example, covalently, hydrophobically, ionically, or combinations thereof. In some embodiments, the binding segment may include a silyl group, e.g., binding segments having a formula:

—Si(R¹)₃₋ₓ(R²)ₓ;

where x=0, 1, or 2;

each group R′ includes independently OH or a hydrolyzable group from among halo, alkoxy, aryloxy, aralkyoxy, and acyloxy; and

each group R² includes independently a non-hydrolyzable group from among alkyl, aryl, and aralkyl.

Generally, the linking group may include any segment suitable for connecting the binding segment with the reactive segment. In illustrative embodiments, the linking segment may comprise alkylene, arylene, or both, and optionally further comprises -NH- or alkyleneoxy, or both.

Generally, the reactive segment may include any segment capable of coupling to one or more analytes such that the analyte may be separated from a sample (e.g., a solution having an analyte dispersed therein). In some embodiments, the reactive segment may include a reactive nitrogen group, e.g., reactive segments having a formula:

—N(R³)₂, or salts thereof;

where each group R³ comprises independently hydrogen, alkyl, aryl, or aralkyl

In some embodiments, the surface-modifying agent may include (aminoethylaminomethyl)phenethyltrimethoxysilane (SIA0588.0, available from Gelest, Inc., Tullytown, PA), N-(2-aminoethyl)-3-aminopropylmethyldimethoxysilane (SIA0589.0, Gelest), N-(2-aminoethyl)-3-aminopropyltrimethoxysilane (SIA0591.0, Gelest), N-(6-aminohexyl)aminopropyltrimethoxysilane (SIA0594.0, Gelest), N-(2-aminoethyl)-11-aminoundecyl-trimethoxysilane (SIA0595.0, Gelest), aminophenyltrimethoxysilane (SIA0599.2, Gelest), N-3[amino(polypropylenoxy)]aminopropyltrimethoxysilane (SIA0599.4, Gelest), 3-aminopropyldimethylethoxysilane (SIA0603.0, Gelest), 3-aminopropylmethyldiethoxysilane (SIA0605.0, Gelest), aminopropylsilanetriol (SIA0608.0, Gelest), 3-aminopropyltriethoxysilane (SIA0610.0, Gelest), 3-aminopropyltrimethoxysilane (SIA0611.0, Gelest), n-butylaminopropyltrimethoxysilane (SIB1932.2, Gelest), N-methylaminopropylmethyldimethoxysilane (SIM6498.0, Gelest), N-methylaminopropyltrimethoxysilane (SIM6500.0, Gelest), N-(trimethoxysilylethyl)- benzyl-N,N,N-trimethylammonium chloride (SIT8395.0, Gelest), 2-(trimethoxysilylethyl)pyridine (SIT8396.0, Gelest), (3-trimethoxysilylpropyl)diethylene- triamine (SIT8398.0, Gelest), and combinations thereof.

Cells used in the current application can be any suitable anchorage dependent cells, for example, mammalian cells, insect cells or plant cells. Suitable mammalian cells can include but not limited to all members of the order Mammalia, such as, for example, human cells, mouse cells, rat cells, monkey cells, hamster cells, and the like. The average seeding density of mammalian cells on buoyant hollow particles can be from 3 to 50, from 3 to 40, from 3 to 30, from 5 to 25, from 10 to 20, or in some embodiments, less than, equal to, or greater than 3, 5, 10, 15, 20, 25, 30, 35, 40, or 50 adherent cells/buoyant hollow particle.

In further embodiments, the present disclosure may relate to a method of culturing mammalian cells. The method may include size fractionating a first volume of buoyant hollow particles to yield a second volume of buoyant hollow particles having a desired particle size distribution. Any conventional size fractionation method may be employed including filtration, decantation, sedimentation, centrifugation, wet or dry screening, air or liquid elutriation, cyclones, static electricity, or combinations thereof. In some embodiments, size fractionating yields a second volume of buoyant hollow particles having a particle size distribution that is narrower than the particle size distribution of the first volume.

Size fractionating may yield a second volume of particles having a particle size distribution with a span of less than about 1, less than about 0.8, less than about 0.7, less than about 0.6, or even less than about 0.5.

Prior to, simultaneous with, or following the step of size fractionating, in some embodiments, the method may further include surface modifying at least a portion of the particles. In some embodiments, surface modifying may include subjecting the particles to an optional pre-treatment step (e.g., to expose or clean a surface of the particle to facilitate surface modification). The optional pre-treatment step may include an alkaline treatment. Alternatively, or additionally, the pretreatment may include an acid or plasma cleaning treatment.

Following any optional pre-treatment step, the method may include providing a plurality of buoyant hollow particles. The method can include contacting the buoyant hollow particles with a media comprising a plurality of mammalian cells and allowing the mammalian cells to attach to the siliceous surface of the buoyant hollow particles.

The method may further include culturing the mammalian cells on the siliceous surface of the buoyant hollow particles by agitation (e.g., inverting, stirring, shaking, etc.), to yield the cell culture to achieve dispersion of the buoyant hollow particles throughout the media. In some embodiments, the media may be agitated such that the buoyant hollow particles are substantially uniformly dispersed throughout the media.

In some embodiments, the method may also include separating the buoyant hollow particles with the mammalian cells attached from the media. Generally, any known methods for separating particles from a meida may be employed. In some embodiments, separating the particles from the media may include allowing the buoyant hollow particles with the mammalian cells attached to float to an upper surface (i.e., air/media interface) of the media.

The buoyant hollow particles of the present disclosure may facilitate rapid separation of the particles from the media. For example, in embodiments in which the particles are separated by flotation to an upper surface of the media, the particles of the present disclosure may separate in less than about 2 minutes, less than about 1 minute, less than about 30 seconds, or even less than about 15 seconds. In this manner, all manner of analytes may be readily captured by the surface-modified particles of the present disclosure. In illustrative embodiments, the method may include capture of one or more proteins and/or one or more nucleic acids from a media utilizing the buoyant hollow particles of the present disclosure.

In some embodiments, the method may also include detaching the mammalian cells from the siliceous surface of the buoyant hollow particles. Generally, any known methods for detaching the mammalian cells from parciles may be employed, for example, by trypsinization or other methods. In some embodiments, the method may also include collecting the detached mammalian cells by sedimentation or centrifugation. Additional cell culture media or additional buoyant hollow particles can be added in the process.

In some embodiments, the compositions and methods of the present disclosure may be useful in, for example, culturing or expanding anchorage-dependent mammalian cells in volume-scalable format.

The following embodiments are intended to be illustrative of the present disclosure and not limiting.

### EMBODIMENTS

1. A composition comprising a plurality of buoyant hollow particles, the buoyant hollow particles comprising a siliceous surface; and a plurality of mammalian cells attached to the siliceous surface of the buoyant hollow particles; wherein the buoyant hollow particles are less dense than a media; and wherein the average seeding density is 3-50 adherent cells/buoyant hollow particle.

2. The composition according to embodiment 1, wherein the buoyant hollow particles comprise glass bubbles.

3. The composition according to any one of embodiments 1-2, wherein the media comprises water.

4. The composition according to any one of embodiments 1-3, wherein the siliceous surface of the buoyant hollow particles are functionalized, coated or treated to provide a surface for growth of mammalian cells.

5. The composition according to any one of embodiments 1-4, wherein the siliceous surface of the buoyant hollow particles are coated with a polymer, a signaling peptide or an antibody or receptor, a surface-modifying agent or a substrate.

6. The composition according to embodiment 5, wherein the surface-modifying agent is bonded to the buoyant hollow particles.

7. The composition according to any one of embodiments 1-6, wherein the buoyant hollow particles have a mean particle size of from 10 vim to 120 vim.

8. The composition according to any one of embodiments 1-7, wherein the buoyant hollow particles have an average density of less than about 1 g/ml.

9. A method for culturing cells, the method comprising providing a plurality of buoyant hollow particles, the buoyant hollow particles comprising a siliceous surface, wherein the buoyant hollow particles are less dense than a media; contacting the buoyant hollow particles with a media comprising a plurality of mammalian cells; allowing the mammalian cells to attach to the siliceous surface of the buoyant hollow particles; and culturing the mammalian cells on the siliceous surface of the buoyant hollow particles by agitation, to yield the cell culture.

10. The method according to embodiment 9, further comprising separating the buoyant hollow particles with the mammalian cells attached from the media.

11. The method according to embodiment 10, wherein the separating step comprises allowing the buoyant hollow particles with the mammalian cells attached to float to an upper surface of the media.

12. The method according to any one of embodiments 9-11, further comprising detaching the mammalian cells from the siliceous surface of the buoyant hollow particles.

13. The method according to any one of embodiments 9-12, further collecting the detached mammalian cells by sedimentation or centrifugation.

14. The method according to any one of embodiments 9-13, further comprising adding additional cell culture media or additional buoyant hollow particles.

15. The method according to any one of embodiments 9-14, where in the average seeding density is 3-50 adherent cells/buoyant hollow particle.

16. The composition according to any one of embodiments 1-2, wherein the media comprises Glucose, FBS and/or DMEM.

The following working examples are intended to be illustrative of the present disclosure and not limiting.

### EXAMPLES

Particle Size Distribution Test Method and Span Value Determination

Particle size distribution of the glass bubbles was measured by light scattering using a laser particle analyzer (Microtrac Incorporated, Mongomeryville, PA). The size for which 10 percent of the particles in the distribution were smaller (10P), the size for which 50 percent of the particles in the distribution were smaller (50P), and the size for which 90 percent of the particles in the distribution were smaller (90P) were determined from the particle size distribution. The span value was calculated from the 10P, 50P and 90P values using the formula, span =(90P-10P)/50P.

Preparation of Buoyant Hollow Particles (Fractionation of K25 Glass Bubbles

K25 glass bubbles (density =0.25 g/mL, mean particle size of 51 microns, span of 1.12) were obtained from the 3M Corporation, Maplewood, MN.

K25 glass bubbles (5 g) and 400 mL of water were added to a cylindrical separatory funnel and mixed by inverting the funnel up and down three times. The separatory funnel was then secured upright for a period (a “fractionation interval”) prior to removal of the liquid phase. During the fractionation interval, the glass bubbles separated into three segments: (i) a top segment of glass bubbles floating at the surface of the water in the funnel; (ii) a bottom segment of fines and shards that settled out of the water; and (iii) a middle segment forming a cloudy suspension of water and glass bubbles between the top and bottom segments. The fractionation interval was selected such that these three distinct segments were apparent and was determined empirically for the desired particle size distribution for a given bubble density and diameter. The fractionation interval was selected to be relatively short (about 1 minute) in order to drain and remove the segments (ii) and (iii). That is, the fractionation period was selected such that it was shorter than the time necessary for the glass bubbles of segment (ii) to rise to the surface of the water in the funnel.

After the fractionation interval, approximately 390 mL of water and glass bubble material from segments (ii) and (iii) was drained from the bottom of the funnel, leaving highly floatable glass bubbles at the surface of the remaining water in the funnel. Water (about 390 mL) was added to the funnel and the process was repeated three more times. The remaining glass bubbles were collected and filtered through a paper filter using an aspirator system. The glass bubbles were rinsed with acetone and the air dried at room temperature.

Using the particle size test method described above, the resulting fractionated K25 glass bubbles were determined to have a mean particle size of 72 microns, a 10P value of 51.9 microns, a 50P value of 68.5 microns, and a 90P value of 96.0 microns. The span value was calculated to be 0.64. The density of the fractionated K25 glass bubbles was assumed to be about the same as the original density of the K25 glass bubbles (0.25 g/mL +/−10%).

Cells and Growth Medium

NIH3T3 GFP cells (AKR-214, Cell Biolabs, Inc. San Diego, CA) were chosen for ease of visualization by microscopy. The cells are maintained as adherent cells in T-flasks. The growth medium was GIBCO DMEM (Dilbecco's Modified Eagle Medium), High Glucose, Pyruvate Medium (#11995040, Thermo Fisher Scientific, Waltham, MA) additionally supplemented with 10% fetal bovine serum (FBS), penicillin (10 U/mL), and streptomycin (10 micrograms/mL) (all of the supplements obtained from Thermo Fisher Scientific).

Shake Flask Preparation

Glass Erlenmeyer flasks (125 mL) were treated with SIGMACOTE siliconizing reagent (Sigma-Aldrich Corporation, St. Louis, MO) according to the manufacturer's instructions to minimize attachment of cells to the culture vessel. The coated flasks were rinsed with deionized water to remove any residuals and then autoclaved to sterilize.

### EXAMPLE 1

Fractionated K25 glass bubbles (0.15 g or 0.2 g) were added to a sterilized, coated Erlenmeyer shake flask. The glass bubbles were sterilized by adding pure ethanol to the flask and shaking the flask in a cell culture incubator (37 ° C./5% CO₂) for ten minutes. The ethanol was removed by allowing the glass bubbles to float to the upper surface of the liquid and aspirating the ethanol from the bottom of the flask. The glass bubbles were then washed with sterile phosphate buffered saline (PBS) pH 7.4 (Thermo Fisher Scientific) three times to remove residual ethanol. Each wash step involved adding 15 mL of PBS to the shake flask, shaking the flask in an orbital motion by hand for approximately 30 seconds, allowing the flask to sit undisturbed, and then aspirating a majority of the PBS from the bottom of the shake flask to leave behind the floating glass bubbles. After the final PBS washing step, 10 mL of the supplemented growth medium (described above) was added to the shake flask. The flask was allowed to shake in the incubator (37 ° C./5% CO₂) until cells were ready to be added.

A T-150 polystyrene flask (Corning Incorporated, Corning, NY) of nearly confluent NIH/3T3 GFP cells was used as the inoculum. The cells were detached from the surface using 0.25% Trypsin-EDTA (Thermo Fisher Scientific). The trypsin was neutralized by adding 20 mL of the supplemented growth medium to the flask and pipetting to wash the cells from the surface of the T-flask (effective trypsinization was verified by light microscopy). The contents of the T-flask were transferred to a sterile 50 mL conical centrifuge tube and the tube was centrifuged (1800g for five minutes) to pellet the cells. The medium was removed from the tube and 15 mL of fresh, supplemented growth medium was added to resuspend the cells. The number of cells was determined using an automated ORFLOW Moxi Z1 cell counter (ORFLO Technologies, Ketchum, ID). A fraction of the trypsinized cells (1.5×10⁷ or 3.0×10⁷ cells) was added to the shake flask containing glass bubbles and growth medium.

The shake flask was placed on an orbital shaker set to 100-120 rpm in an incubator (37° C./5% CO₂). The floating glass bubbles were visibly submerged in the medium during the shaking. The flask was shaken for 2 or 3 days. Samples of the growth medium and glass bubbles were taken using an inverted fluorescence microscope (FITC wavelength, 10x magnification, ECHO Laboratories, San Diego, CA). The obtained images showed that cells were growing attached to the glass bubbles and that the number of free floating cells in the growth medium decreased with time.

After shaking for 2 or 3 days, the cells were harvested from the glass bubbles. The shake flask was removed from the orbital shaker and allowed to sit undisturbed. Most of the glass bubbles with attached cells floated in a layer at the upper liquid surface, but some of the glass bubbles did sink. The glass bubbles with attached cells floating at the upper liquid surface were recovered by using aspiration (pipet connected to a vacuum pump) to remove the majority of the growth medium along with the sunken glass bubbles. A portion of the growth medium was saved to measure the fraction of cells growing in the growth medium (freely floating cells). The remaining glass bubbles were washed by adding 10-15 mL of PBS and shaking the flask in an orbital motion by hand for approximately 30 seconds. The flask was allowed to sit undisturbed. A majority of the PBS was removed by aspiration leaving the layer of glass bubbles that were floating at the upper surface of the liquid. A 3 mL portion of 0.25% Trypsin-EDTA was added and the flask was incubated for three minutes (37 ° C./5% CO₂) with shaking. The trypsin was neutralized by adding 20 mL of the supplemented growth medium to the flask. The entire contents of the flask were transferred into a 50 mL conical centrifuge tube. The tube was centrifuged at 1800g for five minutes to provide a pellet of cells at the bottom of the tube and a layer of glass bubbles floating at the upper liquid surface. Using a pipet, the cell pellet with some residual media was removed from the bottom of the centrifuge tube and then resuspended in a second tube containing supplemented growth medium. A sample of the glass bubble layer was examined by microscopy to verify that the cells were removed from the glass bubbles. The number of cells harvested from the floating glass bubbles was determined using the automated cell counter. Three trials were conducted using different quantities of fractionated K25 glass bubbles, different numbers of added NIH3T3 GFP cells, and different cell harvesting times. The results are presented in Table 1.

TABLE 1

| | Trial 1 | Trial 2 | Trial 3 |
|---|---|---|---|
| Amount of Fractionated | 0.2 | 0.15 | 0.2 |
| K25 Glass Bubbles (g) | | | |
| Number of Cells Added | 3.0 × 10⁷ | 1.5 × 10⁷ | 1.5 × 10⁷ |
| to Flask | | | |
| Days Until Cells | 3 | 2 | 2 |
| Harvested | | | |
| Number of Cells Attached | 1.2 × 10⁷ | 3.0 × 10⁶ | 4.8 × 10⁶ |
| to the K25 Glass Bubbles | | | |
| Number of Cells Recovered | 3.0 × 10⁵ | 1.5 × 10⁶ | 4.8 × 10⁵ |
| in the Growth Medium (i.e. | | | |
| not attached to K25 Glass | | | |
| Bubbles) | | | |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A composition comprising
a plurality of buoyant hollow particles, the buoyant hollow particles comprising a siliceous surface; and
a plurality of mammalian cells attached to the siliceous surface of the buoyant hollow particles;
wherein the buoyant hollow particles are less dense than a media; and
wherein the average seeding density is 3-50 adherent cells/buoyant hollow particle.

2. The composition according to claim 1, wherein the buoyant hollow particles comprise glass bubbles.

3. The composition according to claim 1, wherein the media comprises water.

4. The composition according to claim 1, wherein the siliceous surface of the buoyant hollow particles are functionalized, coated or treated to provide a surface for growth of mammalian cells.

5. The composition according to claim 1, wherein the siliceous surface of the buoyant hollow particles are coated with a polymer, a signaling peptide or an antibody or receptor, a surface-modifying agent or a substrate.

6. The composition according to claim 5, wherein the surface-modifying agent is bonded to the buoyant hollow particles.

7. The composition according to claim 1, wherein the buoyant hollow particles have a mean particle size of from 10 μm to 120 μm.

8. The composition according to claim 1, wherein the buoyant hollow particles have an average density of less than about 1 g/ml.

9. A method for culturing cells, the method comprising providing a plurality of buoyant hollow particles, the buoyant hollow particles comprising a siliceous surface, wherein the buoyant hollow particles are less dense than a media;
contacting the buoyant hollow particles with a media comprising a plurality of mammalian cells;
allowing the mammalian cells to attach to the siliceous surface of the buoyant hollow particles; and
culturing the mammalian cells on the siliceous surface of the buoyant hollow particles by agitation, to yield the cell culture.

10. The method according to claim 9, further comprising separating the buoyant hollow particles with the mammalian cells attached from the media.

11. The method according to claim 10, wherein the separating step comprises allowing the buoyant hollow particles with the mammalian cells attached to float to an upper surface of the media.

12. The method according to claim 9, further comprising detaching the mammalian cells from the siliceous surface of the buoyant hollow particles.

13. The method according to claim 9, further collecting the detached mammalian cells by sedimentation or centrifugation.

14. The method according to claim 9, further comprising adding additional cell culture media or additional buoyant hollow particles.

15. The method according to claim 9, where in the average seeding density is 3-50 adherent cells/buoyant hollow particle.
